(19) United States
(12) Patent Application Publication
(10) Pub. No.: US 2002/0000233 A1
(43) Pub. Date: Jan. 3, 2002

(54) Intravaginal catheter for peineal self-re-education

(75) Inventor: Patrick Jude, Wattignies (FR)

Correspondence Address: Harrison & Egbert, 7th Floor, 412 Main Street, Houston, TX, 77002, US

(73) Assignee: Home Medical Service H.M.S.

(21) Appl. No.: 09/866,222

(22) Filed: May 24, 2001

(30) Foreign Application Priority Data
May 26, 2000 (FR) 00/06.729

(51) Int. Cl. A61B 19/00

(52) U.S. Cl. 128/897

(57) ABSTRACT

The invention relates to an intravaginal catheter for perineal self-re-education. It is constituted by a passive body of revolution (1) and of a determined weight, including:

a rounded introduction end portion (2), extended by,

a cylindrical part (3) defining a main body, the dimensions of which correspond substantially to the ring formed by the perineal muscular system, and which is extended by,

a substantially frustoconical first portion (4), making it possible to achieve control over resistance to the weight of the whole of the object (1), and which is extended by,

a second substantially frustoconical portion (5), flared in a direction opposite that of said first frustoconical portion (4), defining therewith a concave central zone of revolution (6), and which is extended by,

an outer portion forming a ring enabling the object (1) to be gripped with the hand, and preventing the latter from migrating in the vagina.

The present invention relates to an intravaginal catheter for perineal self-re-education.

The object of this re-education is to eliminate different causes of urinary incontinence, on a preventive basis in the postpartum period, or on a curative basis only, or again, as a complement to medical or surgical treatment.

Among the different techniques used in this field, manual re-education, in addition to the levator muscles of the anus, also concerns the bulbospongious muscles. Its principle derives from the exercises recommended by Mr. Kegel in 1948. This re-education is adapted to the results of an initial muscular test. It comprises the following successive conventional phases:

gaining awareness,

learning to appreciate the quality of muscular contraction,

musculation,

effort-based retraining.

Different studies have shown the efficiency of manual re-education as a re-educative technique properly speaking. It appears to be suited to dealing with stress-generated urinary incontinence as it acts on muscular reinforcement of the pelvic floor. Intravaginal work gives good results as regards all the criteria of evaluation.

This technique can be efficiently relayed by self-maintaining exercises, on condition that suitable equipment is available.

Hitherto, only vaginal cones were available. These act by reproducing an endocavitary weight effect which obliges the patient in theory to block her perineal muscular system. In fact, while their use makes it possible to increase the strength of the pelvic muscular floor, it does not enable its strength to be measured objectively. A study of technical literature gives rise to reservations as to the efficiency of this type of equipment.

On the other hand, vaginal cones have the advantage of being used in re-education, hence in self-maintaining exercises, before or after conventional re-education and in the case of women refusing to practise the latter or unavailable to do so.

International patent application WO-98 48 904 discloses a vaginal weight designed to be introduced inside the vagina for the purpose of testing and of examining the striated muscle fibre of the perineal structure. This weight has the particularity of having a weight that is asymmetrically distributed and comprising a weighty element or a plurality of distributed internally weighty elements, with the centre of gravity of the weighty element or the global centre of gravity of the different weighty elements Within the vaginal weight being located below the centre of gravity of said weight when the latter does not contain the weighty element or elements.

Nonetheless, and as aforementioned, such a device does not enable the strength of the pelvic muscular floor to be measured objectively.

In order to obtain such a result, and with the objective of remedying the drawbacks of the known systems, the invention provides an intravaginal catheter for perineal self-re-education, characterised in that it is constituted by a passive solid of revolution of a determined weight, including:

a rounded introduction end portion, extended by,

a cylindrical part defining a main body, the dimensions of which correspond substantially to the ring formed by the perineal muscular system, and which is extended by,

a substantially frustoconical first portion, making it possible to achieve control over resistance to the weight of the whole of the object, and which is extended by,

a second substantially frustoconical portion, flared in a direction opposite that of said first frustoconical portion, defining therewith a concave central zone of revolution, and which is extended by,

an outer portion forming a ring enabling the object to be gripped with the hand, and preventing the latter from migrating in the vagina.

The present invention also relates to the characteristics that will emerge in the course of the description that follows, and which are to be considered separately or according to all of their possible technical combinations.

This description, which has been given by way of a non-limitative example, will make it easier to understand how the invention can be accomplished, with reference to the annexed drawings, wherein:

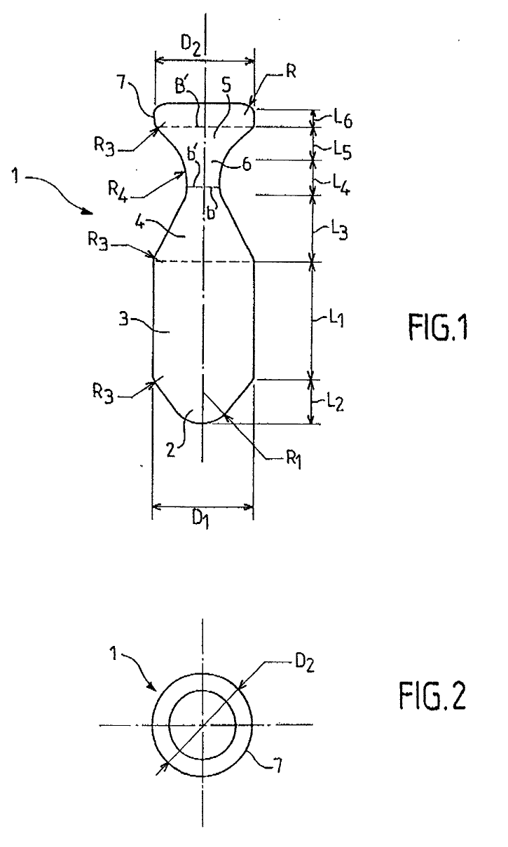

FIG. 1 is a plan view of the intra-vaginal catheter for perineal self-re-education according to the invention;

FIG. 2 is a top view of the outer end of the catheter according to FIG. 1.

The catheter, globally illustrated in FIG. 1, is constituted by a passive body 1 of revolution and of a determined weight, including:

a rounded introduction end portion 2, extended by,

a cylindrical part 3 defining a main body, the dimensions of which correspond substantially to the ring formed by the perineal muscular system, and which is extended by,

a substantially frustoconical first portion 4, making it possible to achieve control over the resistance to the weight of the whole of the object 1, and which is extended by,

a second substantially frustoconical portion 5, flared in a direction opposite that of said first frustoconical portion 4, defining therewith a concave central zone of revolution 6, and which is extended by,

an outer portion forming a ring 7 enabling the object to be gripped with the hand, and preventing the latter from migrating in the vagina.

The dimensions of the endocavitary portion correspond to average anatomical measurements taken, enabling the catheter to be used by all women.

According to an advantageous form of embodiment, introduction portion 2 is in the shape of an ogive, the end of which is designed to have a radius R₁, in particular one of between 8 and 15 mm, and has a length L₂ of approximately 13 mm. It extends towards the cylindrical portion 3 which has a diameter D₁ of approximately 30 mm and a length L₁ of approximately 35 mm.

As to said first frustoconical portion 4, this has a large base B the diameter of which corresponds to the diameter D₁ of cylindrical portion 3 from which it originates. Its small base b has a diameter of approximately 10 mm, for instance.

Said small base b coincides with small base b′ of the second frustoconical portion 5, originating from said first frustoconical portion 4. Large base B′ of said second frustoconical portion 5 has a diameter D₂ of approximately 30 mm, for instance.

First frustoconical portion 4 has a height L₃ of approximately 20 mm, for instance, whereas second frustoconical portion 5 has a height L₅ of approximately 10 mm, for instance.

Said frustoconical portions 4 and 5 define a concave central zone 6 which has, for its part, a height L₄ of approximately 10 mm, for instance, and a radius of curvature R₄ in the order of 12 mm, in particular.

In fact, the double revolute truncated cone, constituted by said portions 4 and 5, in the shape of a diabolo, thus has an overall height of 40 mm, for instance.

As to ring 7 forming the outer portion, this has a diameter D₂ of approximately 30 mm, for instance, merging with large base B′ of said second frustoconical portion 5, from which it originates. It has a thickness L₆ of approximately 5 mm, for instance.

As will be noted from FIG. 1, all the angular zones of Junction of the different portions are rounded to a radius R₃ so as to permit ease of introduction, without either apprehension or risk of injury.

Said ring 7 not only makes it possible, as already mentioned, to grasp the object with the hand, preventing it, moreover, from migrating inside the vagina, but also facilitates the introduction and the extraction of the catheter. In addition, its small thickness enables a user to work in underclothes.

In addition, and this is very important, this same ring 7 prevents the catheter, above all, from abutting on the bony promontory, which would be detrimental to its efficiency.

According to another characteristic of the invention, object 1 has a weight of, for instance, between 350 and 500 grams. The weight function is an essential feature of the catheter and which, in association with frustoconical edge portion 4 extending, for instance, over a length of 20 mm, makes it possible to achieve control over resistance to the weight.

As this catheter is passive, without any external energy being applied, control over resistance to the weight is exercised solely by the patient, which constitutes the sought after perineal self-re-education.

According to another characteristic of the invention, ring 7 forming the outer portion comprises a system for fixing to a counterweight, making it possible to increase the work to be performed.

This particularity is an advantage when the work is carried out or monitored by a therapist who is equipped with a pulling means, the end of which is provided either with a small suction cup or a magnetic end-piece permitting attachment to the outer face of the ring, in order to measure the efforts made and the progress accomplished.

Advantageously, the catheter is made of a metal treated to prevent oxidation and perfectly smooth. For this purpose, use can be made, for example, of drawn chromium steel or stainless steel, or again, of titanium, at least on the surface.

The purpose of this is to guarantee a sterile environment and ease of cleaning. The surface of the catheter has no sharp edges and is perfectly smooth, which makes it easy to introduce without the use of a lubricant, and explains the comfort with which it can be held in place.

The weight of the catheter may cause surprise when it is weighed in one's hand. In reality, the perineal muscles form an extremely strong muscular hammock, and the weight of the catheter is thus perfectly matched to the strength of the muscles.

Furthermore, this weight varies according to the position of the patient and it thus ideally suited to effort-based retraining. Indeed:

in recumbent position, the weight of the catheter is nil;

in sitting position, that is to say at an angle of approximately 90°, the weight of the catheter is divided by two;

in standing position, and solely in this position, the weight of the catheter is exerted downwards, and perineum has to be blocked for it to remain in place.

This instantaneous reduction or increase in the weight of the catheter, according to the patient's position, permits swifter muscular re-education.

The exercises described below follow this progress. The present invention can be implemented as follows.

The patient is examined at the beginning, and then after ten days of exercises, with a visual scale of satisfaction.

1) First Week

Working in recumbent position with the legs stretched out, perfectly relaxed; insert the catheter into the vagina. Start the exercises by placing one hand on the abdominal muscles and the other hand on the lateral face of the buttock muscles to make sure that they play no part. Perineal contraction is maintained by counting aloud to avoid holding one's breath.

1.1) First day “proprioception”

a)—1s contraction—3s rest, Repeat ten times without attempting maximum contraction but scrupulously checking that no part is played by the buttock muscles, the abdominal muscles or pelvic tilt.

Then,

b)—complete rest for 20s. Then repeat exercise a) and then b) for five minutes in all, Perform this exercise morning and evening.

1.2) Second day: “fast muscle fibres”

a) 3s contraction—9s rest Repeat ten times, trying to grip the catheter as firmly as possible and still scrupulously checking that no part 1s played by the buttock muscles, the abdominal muscles or pelvic tilt.

b)—complete rest for 30s. Then repeat exercise a) and then b) for five minutes in all. Perform this exercise morning and evening.

1.3) Third day: “slow muscle fibres”

a)—6s contraction—6s rest. Repeat ten times without attempting to squeeze, at 50% maximum effort, and still scrupulously checking that no part is played by the buttock muscles, the abdominal muscles or pelvic tilt.

b)—complete rest for 30s. Then repeat exercise a) and then b) for five minutes in all. Perform this exercise morning and evening.

1.4) Continue the exercises morning and evening alternating the “fast fibre” and “slow fibre” exercises up to the end of the first week, that is to say:

“fast fibre” exercises on even days, 2, 4, 6

“slow fibre” exercises on odd days, 3, 5, 7.

2) Second Week

Working in sifting position, relaxed, with the back supported, the legs stretched out, one hand on the abdominal muscles and the other on the lateral face of the buttock muscles to make sure that they play no part. Perineal contraction is maintained by counting aloud to avoid holding one's breath.

2.1) First, second and third days:

Repeat the same exercises, and in the same chronological order as for the first three days, but in sitting position.

The sitting position makes it possible to vary the weight of the catheter ad infinitum and to program the intensity of the exercise. By modifying the sitting angle between 0° and 90°, it is possible to vary the weight, hence the efficiency, of the device. The easiest position is the one closest to 0°, and the most difficult the one at 90°, as perineal contraction is required to overcome the weight of the catheter.

2.2) Continue the exercises in sitting position morning and evening, alternating the “fast fibre” and “slow fibre” exercises up to the end of the week, that is to say:

“fast fibre” exercises on even days, 2, 4, 6

“slow fibre” exercises on odd days, 3, 5, 7.

3) Third Week:

Working in standing position, with the catheter in place in the vagina. There is no longer any need to position the hands or count aloud as the patient now knows how to control herself and avoid parasitical gestures.

The shape of the catheter enables it to be used when wearing underclothes, hence when performing a routine household task, for example.

3.1) First day: “learning how to block muscles when making brief efforts” with the catheter in place.

a)—perineal contraction—1 cough, blocking the perineum, standing position with legs in normal position—10s rest. Repeat the exercise ten times;

b)—30s complete rest;

c)—perineal contraction—5 successive coughs, blocking the perineum, standing position with legs in normal position—20s rest. Repeat the exercise ten times;

d)—complete rest for 30s;

e)—perineal contraction—1 cough, blocking the perineum, standing position with legs astride—10s rest. Repeat the exercise ten times;

f)—complete rest for 30s;

g)—perineal contraction—5 successive coughs, blocking the perineum, standing position with legs astride—20s rest. Repeat the exercise ten times;

3.2) Perform this exercise morning and evening for seven days.

4) Fourth Week:

“learning how to block muscles when making prolonged efforts” with the catheter in place.

a)—perineal contraction—squatting once, blocking the perineum—10s rest. Perform the exercise ten times:

b)—complete rest for 30s.

Perform exercise a) and then b) for 10 minutes in all.

And then:

c)—perineal contraction—squat 3 times in succession, blocking the perineum—20s rest. Repeat the exercise ten times.

Perform this exercise morning and evening.

5) Twenty-ninth Day:

“initial testing”: holding the catheter in place while performing habitual activities for half an hour.

6) Thirtieth Day:

“final testing”: holding the catheter in place while performing habitual activities for half an hour. At this stage, the exercises can be performed while doing the housework, washing up, vacuuming, etc. The catheter, blocked in position by the perineum, stays in place when one climbs the stairs, when in squatting position, etc.

The aim is accomplished at the end of re-education, the object of which is to achieve blocking of the perineum in standing position without effort. A re-educated, efficient perineum contracts automatically in standing position when an effort is made, thus making it possible to eliminate urinary incontinence.

1. Intravaginal catheter for perineal self-re-education, characterised in that it is constituted by a passive body of revolution (1) and of a determined weight, including:
a rounded introduction end portion (2), extended by,
a cylindrical part (3) defining a main body, the dimensions of which correspond substantially to the ring formed by the perineal muscular system, and which is extended by,
a substantially frustoconical first portion (4), making it possible to achieve control over resistance to the weight of the whole of the object 1, and which is extended by,
a second substantially frustoconical portion (5), flared in a direction opposite that of said first frustoconical portion (4), defining therewith a concave central zone of revolution (6), and which is extended by,
an outer portion forming a ring enabling the object (1) to be gripped with the hand, and preventing the latter from migrating in the vagina.

2. Intravaginal catheter according to claim 1, in which the introduction portion (2) is in the shape of an ogive the end of which designed to have a radius R₁, of between 8 and 15 mm.

3. Intravaginal catheter according to claim 1, in which the cylindrical portion (3) has a diameter D₁ of approximately 30 mm and a length L₁ of approximately 35 mm.

4. Intravaginal catheter according to claim 1, in which said first frustoconical portion (4) has a large base B the diameter of which corresponds to that, D₁, of the cylindrical portion (3) from which it originates, a height L₃ of approximately 10 mm, and a small base b of approximately 10 mm, coinciding with the small base b′ of said second frustoconical portion (5), the large base B′ of which has a diameter D₂ of approximately 30 mm also, said frustoconical portions (4,5) defining said concave central zone (6) having a height L₄ of approximately 10 mm.

5. Intravaginal catheter according to claim 1, in which the ring (7) forming the outer portion has a diameter D₂ of approximately 30 mm, merging with the large base B′ of said second frustoconical portion (5), and a thickness L₆ of approximately 5 mm.

6. Intravaginal catheter according to any one of claims 1 to 5, in which the object (1) has a weight of between 350 and 500 grams.

7. Intravaginal catheter according to any one of claims 1 to 6, in which the object (1) is made of a metal treated to prevent oxidation and perfectly smooth.

8. Intravaginal catheter according to any one of claims 1 to 7, in which the ring (7) forming the outer portion comprises a system for fixing to a counterweight making it possible to increase the work performed.
